(19) United States
(12) Patent Application Publication
(10) Pub. No.: US 2012/0318262 A1
(43) Pub. Date: Dec. 20, 2012

(54) Pyrvinium Wound Treatment Methods and Devices

(76) Inventors: Ethan Lee, Brentwood, TN (US); Pampee Paul Young, Brentwood, TN (US)

(21) Appl. No.: 13/426,210

(22) Filed: Mar. 21, 2012

Related U.S. Application Data
(60) Provisional application No. 61/454,817, filed on Mar. 21, 2011.
(60) Provisional application No. 61/454,821, filed on Mar. 21, 2011.

(51) Int. Cl. A61K 31/4709; A61M 1/00; A61N 1/36; A61M 37/00; A61P 17/02; A61K 9/70; A61K 39/395; A61K 38/48; A61K 38/00; A61K 35/00; A61K 35/64; A61K 33/00; A61G 10/00; A61K 38/18

(52) U.S. Cl. 128/202.17; 604/313; 607/3; 604/20; 514/314; 514/7.6; 424/133.1; 424/94.63; 514/2.4; 424/93.1; 424/537; 424/718; 424/445; 424/446

(57) ABSTRACT

The present invention concerns a pyrvinium compound or salt or an analog thereof for the treatment of wounds. The present invention also concerns devices for delivering a pyrvinium compound or salt or an analog thereof for a wound.

This application claims benefit of priority to U.S. Provisional Application Ser. Nos. 61/454,817 and 61/454,821, both filed Mar. 21, 2011, the entire contents of both applications being hereby incorporated by reference.

### BACKGROUND OF THE INVENTION

1. Field of the Invention

The present invention relates generally to the fields of medicine and biology. More particularly, it concerns the use of a pyrvinium compound, or salts or analogs thereof, in the treatment of wounds.

2. Description of Related Art

The Wnt family of developmental genes has been shown to be involved in proliferation, differentiation, and cell to cell signaling during embryogenesis. In addition, several Wnt genes have been shown to be expressed during carcinogenesis. Also, certain Wnt genes involved in the canonical pathway (Wnt/β-catenin pathway) have been implicated in wound-healing processes. For example, Wnt-4 gene expression is found in mouse wounds from 2 hours to 30 hours post-wounding and is also stimulated by direct trauma to murine fibroblasts in culture. Expression is greatly enhanced by the addition of a short plasmin digest of fibrin. Therefore the regulation of Wnt-4, appears to be complex, with expression being stimulated both by direct trauma and by the influence of clotting and fibrinolysis products (Labus et al., 1998). Fathke et al. (2006) have shown that ectopic activation of β-catenin-dependent Wnt signaling with lithium chloride in the wound resulted in epithelial cysts and occasional rudimentary hair follicle structures within the epidermis. In contrast, forced expression of Wnt-5a in the deeper wound induced changes in the interfollicular epithelium mimicking regeneration, including formation of epithelia-lined cysts in the wound dermis, rudimentary hair follicles and sebaceous glands, without formation of tumors.

Despite these reports, the Wnt pathway's involvement in wound repair remains an unexploited therapeutic approach.

### SUMMARY OF THE INVENTION

Thus, in accordance with the present invention, there is provided a method of treating a wound in a subject comprising contacting the wound with a composition comprising pyrvinium or a salt or analog thereof. The wound may be a dermal wound, an epidermal wound, a burn, a laceration or abrasion, an infectious lesion, a surgical site, an ulcer, a puncture, a chronic wound, a scar, such as a hypertrophic scar, a keloid or a blister. The subject may be a human, a non-human mammal a reptile or a bird.

The pyrvinium or a salt or analog thereof may be contacted with the wound in a wound dressing, in a gel, salve or ointment, in a topical spray, in a powder, by injection local or regional to the wound, in a topical liquid or with a suture. The method may further comprise providing to the subject a second wound therapy, such as hyperbaric oxygen therapy (HBO), negative pressure therapy (VAC), electrical stimulation, phototherapy or acoustic stimulation. The second wound therapy may alternatively be a corticosteroid, a cytotoxic drug, an antibiotic, an antiseptic, nicotine, an anti-platelet drug, an NTHE, colchicine, an anti-coagulant, a vasoconstricting drug or an immunosuppressive, a growth factor, an antibody, a protease, a protease inhibitor, an antibacterial peptide, an adhesive peptide, a hemostatic agent, living cells, honey, or nitric oxide, such as those embedded in a wound dressing.

In another embodiment, there is provided a method of promoting wound tissue hemostasis in a subject comprising contacting the wound with a composition comprising pyrvinium or a salt or analog thereof. In yet another embodiment, there is provided a method of promoting wound tissue proliferation in a subject comprising contacting the wound with a composition comprising pyrvinium or a salt or analog thereof. In still yet another embodiment, there is provided a method of promoting wound tissue contraction in a subject comprising contacting the wound with a composition comprising pyrvinium or a salt or analog thereof. In a further embodiment, there is provided a method of promoting wound tissue remodeling in a subject comprising contacting the wound with a composition comprising pyrvinium or a salt or analog thereof. In still a further embodiment, there is provided a method of reducing wound tissue scarring in a subject comprising contacting the wound with a composition comprising pyrvinium or a salt or analog thereof.

Another embodiment comprises a device for the treatment of a wound in a subject comprising (a) a composition comprising pyrvinium or a salt or analog thereof; (b) a sterile dressing into or onto which the pyrvinium, salt or analog is disposed. The sterile dressing may be a compression dressing or a non-adherent dressing. The device may further comprise one or more of a lubricant, an absorber, a sponge, a wound veil, an odor control agent, and/or a cover. The pyrvinium, salt or analog may be contained in a liquid, salve, ointment, gel or powder disposed in or on the sterile dressing. The sterile dressing may be a film, foam, semi-solid gel, pad, gauze, fabric. It may also be a silicone dressing, a fibrin/fibrinogen dressing, a polyacrylamide dressing, a PTFE dressing, a PGA dressing, a PLA dressing, a PLGA dressing, a polycaprolactone dressing or a hyaluronic acid dressing.

The sterile dressing may further comprise gelatin, silver, cellulose, an alginate, collagen, a hydrocolloid, a hydrogel, a skin substitute, a wound filler, a growth factor, an antibody, a protease, a protease inhibitor, an antibacterial peptide, an adhesive peptide, a hemostatic agent, living cells, honey, or nitric oxide. The sterile dressing may further comprises one or more of a corticosteroid, a cytotoxic drug, an antibiotic, an antimicrobial, an antifungal, an antiseptic, nicotine, an anti-platelet drug, an NSAID, colchicine, an anti-coagulant, a vasoconstricting drug or an immunosuppressive. The device may further comprise a substance or element for the fixation of the device to a wound, such as an adhesive or a bandage.

The device may, further comprise a port providing operable connection between the sterile dressing and a tube, including where a cover is provided for an airtight seal to or around a wound surface. The device may further comprise a drainage tube operably connected to the port at one end and suitable for attachment to a negative pressure device at another end. The sterile dressing in this embodiment may be gauze or foam.

Also provided is a method of promoting wound repair in a subject comprising contacting the wound with a device as described above. The method may further comprise applying negative pressure to the wound, may further comprise applying hyperbaric oxygen therapy to the wound, may further comprise electrical stimulation, may further comprise phototherapy, and may further comprise acoustic stimulation.

The wound may be a dermal wound, an epidermal wound, a burn, a laceration or abrasion, an infectious lesion, a surgical site, an ulcer, a puncture, a chronic wound, a scar, such as a hypertrophic scar, a keloid or a blister. The subject may be a human or a non-human mammal, a reptile, or a bird.

Also provided is a suture comprising pyrvinium or a salt or analog thereof impregnated into or disposed thereon. The suture may be an absorbable suture or a liquid suture.

It is contemplated that any method or composition described herein can be implemented with respect to any other method or composition described herein.

The use of the word “a” or “an” when used in conjunction with the term “comprising” in the claims and/or the specification may mean “one,” but it is also consistent with the meaning of “one or more,” “at least one,” and “one or more than one.”

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1. Schematic of Wnt canonical (β-catenin) and non-canonical signaling pathways.

FIGS. 2A-B. (FIG. 2A) Pyrvinium inhibits TOPflash activation with an IC₅₀ of ˜10 nM. HEK 293 STF (TOPflash) reporter cells were treated with Wnt3a-conditioned media and/or pyrvinium for 24 hours. Graph represents mean±s.e.m of TOPflash signal normalized to cell number (performed in quadruplicate). A structurally related compound, Cmpd 211 fails to inhibit TOPflash. (FIG. 2B) Pyrvinium decreases levels of endogenous Wnt target genes AXIN2 and c-MYC. Data shown represent mean of four independent amplification reactions, graphed as relative expression to unstimulated cells. Expression levels were normalized to β-actin mRNA. Error bars, RQ values with 95% confidence.

FIGS. 3A-C. CK1α is the intracellular target of pyrvinium. (FIG. 3A) Pyrvinium stimulates the phosphorylation of β-catenin in vitro. A kinase reaction was assembled in vitro with purified Axin, β-catenin, GSK3, and CK1 (100 nM each) in the absence or presence of pyrvinium (10 nM). Phosphorylation of β-catenin on GSK3 sites (p33,37,41) and the priming CK1α site (p45) was detected by immunoblotting. (FIG. 3B) Pyrvinium binds and activates CK1α but not kinases representative of other major branches of the kinome. Pyrvinium (10 nM) was incubated with purified recombinant kinases, and binding and kinase activities were assessed. Ligand-binding is based on the innate fluorescent property of pyrvinium and nitrocellulose immobilized protein. (FIG. 3C) Downregulating CK1α blocks the transcriptional responses to pyrvinium. A Jurkat cell line with inducible shRNA for CK1α (CK1α^{sh}) was incubated with pyrvinium (30 nM). Cells were treated with Wnt3a-conditioned media and lysates were assayed for TOPflash to assess Wnt signaling.

FIGS. 4A-F. Identification and organizational activity of granulation tissue as demonstrated in H&E stained slides generated in PVA sponges injected with pyrvinium (FIG. 4A) or Cmpd 211 (FIG. 4B). Representative sections stained with anti-CD31 (PECAM-1) to assess vascular density of sponge granulation tissue treated with pyrvinium (FIG. 4C) or Cmpd 211 (FIG. 4D). (FIG. 4E) Morphometric analysis of vascular density by anti-CD31 immunohistochemistry. Pictures from three fields of view from each section were assessed from four animals. The fraction of the field area positive for each was determined by point counting the total area comprising positive pixels divided by total area containing nucleated cells and the averages were graphed. (FIG. 4F) Graphed morphometric analysis of number of Ki-67+ cells (per high power; 40×) in pyrvinium or Cmpd 211 treated granulation tissue.

FIGS. 5A-D. Mouse MI model. Representative mouse EKG tracings pre (FIG. 5A) and post (FIG. 5B) coronary artery ligation. (FIG. 5C) Photomicrograph of Masson trichrome stain of a murine heart at 30 d showing a large area of transmural replacement fibrosis (infarction). Arrows mark lateral and posterior left ventricle (LV). LVIDD, LVIDS=LV internal diameter at systole and diastole, respectively. IVSS, IVSD=interventricular septal distance at systole and diastole, respectively. RV=right ventricle. (FIG. 5D) Graph of percent difference of LVIDD, LVIDS, IVSS and IVSD at d 7 and d 30 after treatment with a single (25 ml) injection of pyrvinium (n=22 animals injected; n=7 survived) or Cmpd 211 (n=6 injected; n=6 survived). A single injection of pyrvinium resulted in favorable remodeling of the mouse myocardium following infarction. Cardiac dimensions were obtained from 2-D guided M-mode images (100 frames/sec) and were read blinded using short axis and a parasternal long-axis views with the leading edge method. All echo measurements were averaged over 3 consecutive beats on unsedated mice at 7 and 30 days after infarction. Statistical significance assessed by the Wilcoxon rank sum test.

FIGS. 6A-F. Pyrvinium induces cardiomyocyte mitosis. Ki-67 positive nuclei in mouse heart evident within the scar and peri-infarct tissue (FIG. 6A) or remote myocardium (FIG. 6B) were quantified. *p<0.05 using One way ANOVA with Newman-keuls post-test. Representative immunostained sections of remote myocardium using anti-Ki-67 to show more mitotic nuclei in pyrvinium (FIG. 6C) vs. Cmpd 211- (FIG. 6D) treated hearts. (FIG. 6E) pH3-positive mononucleated, differentiated cardiomyocyte in pyrvinium-treated remote myocardium but not in Cmpd 211-treated tissue (FIG. 6F).

FIG. 7. Pyrvinium application results in ear wound repair. Right ear of mouse receives pyrvinium treatment. Reduced hole shows enhanced wound repair.

FIGS. 8A-F. Histologic evaluation shows Pyrvinium effects regenerative repair of ear wounds. Representative photomicrographs of histologic sections of mice ears treated with control compound 211 (FIGS. 8A-B) or pyrvinium (FIGS. 8C-F). (FIG. 8A) Low magnification (4×) view of H&E stained slide showing epithelial repair of wounded skin treated with 211 at 30 days after injury. The 2 mm hole is evident. (FIG. 8B) High magnification (20×) H&E of compound 211-treated ears showing hypertrophic epithelium. (FIGS. 8C-D) represent H&E of low (10×) and high (20×) magnification view of pyrvinium-treated ears. (FIGS. 8E-F) Trichrome blue staining showing low (FIG. 8E, 10×) and high power (FIG. 8F, 20×) view of pyrvinium-repaired ear.

FIGS. 9A-F. Activation of intracellular CK1α by small molecules inhibits Wnt signaling. (FIG. 9A) Pyrvinium activates CK1α in cultured cells. HEK 293 cells overexpressing CK1α were treated with pyrvinium pamoate (100 nM). CK1α immunoprecipitated from lysates was incubated with purified β-catenin (100 nM) in kinase assay containing (^{γ32}P)ATP. Samples were processed for SDS-PAGE/autoradiography. As control, immunoprecipitates were treated with the CK1 inhibitor, CKI-7 (1 μM). Immunoblots of β-catenin and CK1α show equivalent amounts of protein were used in each sample. (FIG. 9B) Pyrvinium co-immunoprecipitates with CK1α. HEK 293 cells expressing HA-GSK3, HA-CK1α, or HA alone (vector control) were treated with pyrvinium pamoate (100 nM). HA immunoprecipitates were analyzed by LCMS. Graph represents mean±s.e.m. of relative abundance of pyrvinium normalized to bead control (performed in triplicate). Immunoblots show that comparable amounts of HA-tagged proteins and light chain IgG (LC-IgG) were present in the immunoprecipitates. (FIGS. 9C-F) Derivatives of pyrvinium that inhibit Wnt signaling also activate CK1α. (FIG. 9C) Structure of VU-WS113. (FIGS. 9D-E) HEK 293 STF cells were treated with Wnt3a and the indicated concentrations of VU-WS113 (FIG. 9D) or VU-WS211 (FIG. 9E) and assayed for luciferase activity. Mean±s.e.m. of TOPflash activity normalized to cell number is shown (performed in quadruplicate and displayed as percent response). (FIG. 9F) CK1α (100 nM) was incubated with recombinant casein (100 nM) in the presence or absence of compounds in a kinase reaction containing [γ32P]ATP. Samples were analyzed by SDS-PAGE/autoradiography and the extent of ³²P incorporation into casein (in the presence of pyrvinium relative to vehicle) assessed. Mean±s.e.m. is shown (performed in triplicates).

FIG. 10. Mouse ear treated with compound 113 results in repair. Representative photomicrograph of histologic section of a mouse ear treated with 113 (magnification 4×). Injured hole was repaired. Tissue proliferation around cartilage was noted.

FIGS. 11A-C. C-113 (pyrvinium analog) significantly enhances post-infarct proliferation of cardiac progenitors (FIG. 11A) Immunostaining using anti-Ki67 (marker for proliferation) in representative paraffing sections of periinfarct region of LV of murine hearts from control (C-211) or C-113 treated animals 7 d after injury. The right most panel shows higher magnification of the boxed area. (FIG. 11B) Immunofluorescent colocalization of CPC marker, Sca1 (green) and cell proliferation marker, Ki67 (red) and the merge (right panel). (FIG. 11C) The average numbers of Ki67+ cells that are both Sca1+ or Sca1− identified by co-immunofluorescent analysis shown in “B” of histologic LV tissue.

### DESCRIPTION OF ILLUSTRATIVE EMBODIMENTS

As discussed above, the involvement of Wnt signaling in wound repair has been known for some time, but to date the exploitation of this pathway in promoting wound healing has yet to be achieved. The present inventors have previously described Wnt-modulating effect of the FDA approved compound pyrvinium, and suggested that it might be able to aid in wound repair (see U.S. Patent Publication 2009/0099062). Until recently, however, no evidence for such an effect had been shown. Now, the inventors show that in a cardiac model, wound repair is in fact promoted by pyrvinium. More notable, in the context of “external” tissue damage, such as that incurred on skin or mucosal surfaces, pyrvinium shows a surprising ability to promote wound healing. These and other aspects of the invention are described in greater detail below.

### I. Wnt PATHWAY SIGNALING

The wnt signaling pathway describes a complex network of proteins most well known for their roles in embryogenesis and cancer, but also involved in normal physiological processes in adult animals. Wnt proteins form a family of highly conserved secreted signaling molecules that regulate these interactions. Insights into the mechanisms of Wnt action have emerged from several systems: genetics in Drosophila and Caenorhabditis elegans; biochemistry in cell culture and ectopic gene expression in Xenopus embryos. Many Wnt genes in the mouse have been mutated, leading to very specific developmental defects. As currently understood, Wnt proteins bind to receptors of the Frizzled and LRP families on the cell surface. Through several cytoplasmic relay components, the signal is transduced to β-catenin, which then enters the nucleus and forms a complex with TCF to activate transcription of Wnt target genes.

The name Wnt was coined as a combination of Wg (“wingless”) and Int. The wingless gene had originally been identified as a segment polarity gene in Drosophila melanogaster that functions during embryogenesis. and also during adult limb formation during metamorphosis. The INT genes were originally identified as vertebrate genes near several integration sites of mouse mammary tumor virus (MMTV). The Int-1 gene and the wingless gene were found to be homologous, with a common evolutionary origin evidenced by similar amino acid sequences of their encoded proteins.

Mutations of the wingless gene in the fruit fly were found in wingless flies, while tumors caused by MMTV were found to have copies of the virus integrated into the genome forcing overproduction of one of several Wnt genes. The ensuing effort to understand how similar genes produce such different effects has revealed that Wnts are a major class of secreted morphogenic ligands of profound importance in establishing the pattern of development in the bodies of all multicellular organisms studied.

The Wnt pathway involves a large number of proteins that can regulate the production of Wnt signaling molecules, their interactions with receptors on target cells and the physiological responses of target cells that result from the exposure of cells to the extracellular Wnt ligands. Although the presence and strength of any given effect depends on the Wnt ligand, cell type, and organism, some components of the signaling pathway are remarkably conserved in a wide variety of organisms, from Caenorhabditis elegans to humans. Protein homology suggests that several distinct Wnt ligands were present in the common ancestor of all bilaterian life, and certain aspects of Wnt signaling are present in sponges and even in slime molds.

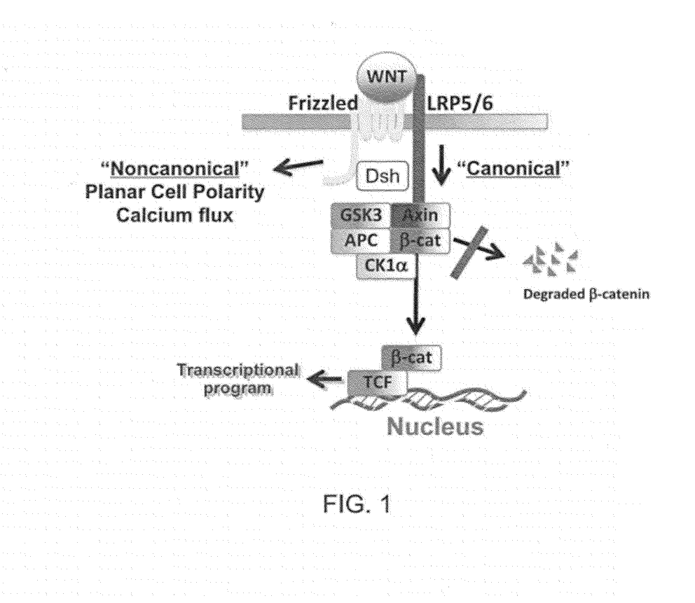

Wnt ligands bind their cognate Frizzled-family receptors to regulate diverse biological processes including cell adhesion, proliferation, migration, and differentiation (Reya and Clevers, 2005; Widelitz, 2005; Shtutman et al., 1999; Tahinci and Lee, 2004). The central player of the “canonical” (Wnt/β-catenin) pathway is β-catenin, which is maintained at a low level in the cytoplasm by its association with a complex (axin, APC, GSK3β) that promotes its phosphorylation and targeted destruction (FIG. 1) (Reya and Clevers, 2005; Widelitz, 2005; Shtutman et al., 1999; Tahinci and Lee, 2004). Upon binding of Wnt by Frizzled and the LRP5 or 6 coreceptor (LRP5/6), the destruction complex is inhibited; β-catenin accumulates and translocates to the nucleus where it interacts with the Tcf/Lef1 transcription factors to regulate Wnt-specific gene expression (Reya and Clevers, 2005; Widelitz, 2005; Shtutman et al., 1999; Tahinci and Lee, 2004). “Non-canonical” Wnt signaling (e.g. planar cell polarity and intracellular calcium release (Reya and Clevers, 2005; Widelitz, 2005; Shtutman et al., 1999; Tahinci and Lee, 2004)) is less well understood at the molecular level.

Wnt signaling has been shown to be a major regulator of cardiogenesis (Cleutjens et al., 1999; Foley and Mercola, 2005; Salloway, 2003). Prior to gastrulation, Wnt/β-catenin signaling promotes cardiac differentiation whereas signaling during gastrulation inhibits heart formation (Cleutjens et al., 1999; Foley and Mercola, 2005; Salloway, 2003). Consistent with these studies, early treatment of mouse embryonic stem cells with Wnt3a stimulates mesoderm induction whereas late Wnt3a stimulation inhibits cardiac differentiation. Furthermore, the Wnt inhibitors Dickkopf-1 (Dkk-1) and secreted frizzled-related proteins (sFRPs) have been shown to induce cardiac differentiation of stem cells (Cleutjens et al., 1999; Salloway, 2003; Pandur et al., 2002)). Although these studies clearly demonstrate the importance of Wnt signaling in cardiac development, less is known about its role in adult cardiac repair. A recent study using Wnt (axin2-LacZ) reporter mice demonstrated that Wnt signaling is increased post-MI in cardiomyocytes of the border zone and remote area between 7-21 days whereas infiltrating CD45⁺ inflammatory cells showed Wnt activation between 3-7 days (Oerlemans et al., 2009). Hence, endogenous activation of the Wnt pathway occurs in the heart in cardiomyocytes and other heart cells and is evident just prior to the initiation of the remodeling phase (day 10-26) of murine infarct repair. The inventors hypothesize that infarct-induced Wnt activation contributes to adverse cardiac remodeling, a process that may be averted by Wnt inhibition. Several recent studies support this hypothesis. Transgenic mice in which β-catenin was downregulated in an alpha-MHC-restricted manner (i.e., resulting in lower cardiac Wnt signaling) demonstrated favorable ischemic remodeling (Zelarayàn et al., 2008). Other groups reported functional deterioration after injury in mice expressing a stabilized β-catenin (i.e., activated Wnt signaling) in cardiomyocytes (Malekar et al., 2010; Baurand et al., 2007). Finally, the inventors and others have shown that mesenchymal stem cells overexpressing sFRP2, a Wnt inhibitor, reduced cardiomyocyte apoptosis (Mirotsou et al., 2007; Alfaro et al., 2008).

Several antagonists of the Wnt pathways have been characterized (Kawano and Kypta, 2003). One class, including sFRPs, binds and sequesters Wnts to inhibit both canonical and non-canonical Wnt signaling (Kawano and Kypta, 2003). Fusion of Frizzled8-cysteine rich domain (binds Wnt) to the human Fc domain inhibited Wnt signaling and teratocarcinoma growth in mice but has not been widely used in vivo possibly due to its low in vivo efficacy or issues of selectivity (DeAlmeida et al., 2007). The Dick class inhibits canonical Wnt signaling by binding to LRP5/LRP6 of the Wnt receptor complex ((Kawano and Kypta, 2003). Recently a novel class of small molecule Wnt inhibitors has been identified that act by inhibiting tankyrase, a poly(ADP-ribose) polymerase (Chen et al., 2009; Huang et al., 2009). These compounds have not been shown to be effective in vivo, possibly due to toxicity and/or bioavailability. The inventors previously identified a FDA-approved drug, pyrvinium, as a potent inhibitor of Wnt signaling that acts by binding and activating casein kinase 1α. It is this molecule that the inventors have now explored further and determined to be a highly effective wound-healing agent.

### II. PYRVINIUM COMPOUNDS AS THERAPEUTIC AGENTS

A. Properties and Synthesis

The present invention relates to pyrvinium, or 6-(Dimethylamino)-2-[2-(2,5-dimethyl-1-phenyl-1H-pyrrol-3-yl)ethenyl]-1-methylquinolinium, C₂₆H₂₈N₃ (U.S. Pat. No. 2,925,417), or salt, or an analog thereof that demonstrates similar wound healing properties. Pyrvinium was historically been used in the treatment of enterobiasis caused by Enterobius vermicularis (pinworm). However, pyrvinium has generally been replaced by other anthelmintics (e.g., mebendazole or pyrantel). It appears to prevent the parasite from utilizing exogenous carbohydrates. Here, pyrvinium is shown to promote wound healing and repair. Thus, the present invention provides pyrvinium or an analog thereof as a therapeutic agent for treating a variety of tissue injuries, including particularly those occurring on the skin and on mucosal tissues.

The pyrvinium analog (VU-WS113) is produced starting with N-(6-bromoquinolin-2-yl)-2,5-dimethyl-1-phenyl-1H-pyrrole-3-carboxamide, which is prepared as follows. In a conical shaped microwave vial was added 2-amino-6-bromoquinoline hydrochloride (100 mg, 0.385 mmol) and dichloroethane (DCE, 3.8 mL). To this suspension was added Hunig's base (0.30 mL, 1.73 mmol). The solution turned deep brown and the solids dissolved. 2,5-dimethyl-1-phenyl-1H-pyrrole-3-carboxylic acid (107 mg, 0.501 mmol) and 1-[chloro(pyrrolidin-1-yl)methylene]pyrrolidin-1-ium hexafluorophosphate (V) (PyClU, 256 mg, 0.771 mmol) were added. The microwave vial was capped and heated under microwave irradiation for 1.5 h at 110° C. After cooling, the solution was concentrated and the residue was purified on silica gel using a Biotage SNAP cartridge (2% to 20% MeOH in dicholormethane) to yield 144 mg (0.34 mmol, 89%) of the desired compound as a beige powder. LCMS: RT=1.44 min, >90% @ 254 nm, >92% @ 220 nm; m/z (M+1)+=420. ¹H NMR (400 MHz, CD3OD, δ (ppm)): 8.6 (d; J=9.2 Hz; 1H), 8.0 (d; J=9.2 Hz; 1H), 7.9 (s; 1H), 7.8 (s; 1H), 7.5-7.4 (m; 4H), 7.2 (d; J=6.8 Hz; 2H), 6.3 (s; 1H), 2.4 (s; 3H), 2.0 (s; 3H). ¹³C NMR (100 MHz; CD₃OD, δ (ppm)): 164.4, 152.4, 145.5, 137.6, 137.3, 136.5, 133.2, 129.7, 129.6, 129.4, 129.0, 128.9, 128.2, 127.3, 118.1, 115.6, 133.8, 104.9, 12.9, 12.7. HRMS calculated for C22H19N3OBr (M+H)+ m/z: 420.0711, measured 420.0707.

The compound 2,5-dimethyl-N-(6-(morpholinomethyl)quinolin-2-yl)-1-phenyl-1H-pyrrole-3-carboxamide (VU-WS113) is prepared as follows. In a conical shaped microwave vial was added N-(6-bromoquinolin-2-yl)-2,5-dimethyl-1-phenyl-1H-pyrrole-3-carboxamide (71 mg, 0.169 mmol), potassium trifluoro(morpholinomethyl)borate (70 mg, 0.338 mmol), 2-Dicyclohexylphosphino-2′,4′,6′-triisopropylbiphenyl (XPhos, 16.1 mg, 0.034 mmol), cesium carbonate (165 mg, 0.507 mmol), and palladium (II) acetate (3.8 mg, 0.017 mmol), THF (0.63 mL) and water (0.06 mL). The microwave vial was capped and the solids were stirred at RT for 5 min to aid in dissolution. Once a clear solution was observed, the vial was heated to 80° C. for 10 min followed by heating to 145° C. for 45 min. After cooling, the solution was diluted with dichloromethane and dried over MgSO₄. The solution was filtered, concentrated and purified on silica gel using a Biotage SNAP cartridge (2% to 20% MeOH in dichloromethane) to yield 71 mg (0.16 mmol, 95%) of VU-WS113 as a yellow oil: LCMS: RT=1.11 min; m/z (M+1)+=441. ¹H NMR (400 MHz, CDCl3, δ (ppm)): 8.4 (d; J=8.8 Hz; 1H), 8.3 (d; J=8.8 Hz; 1H), 7.8-7.7 (m; 5H), 7.3 (d; J=7.2 Hz; 2H), 6.5 (s; 1H), 3.8-3.7 (m; 10H), 2.5-2.4 (m; 8H), 2.3 (s; 3H), 2.0 (s; 3H). ¹³C NMR (100 MHz; CDCl3, δ (ppm)): 166.7, 152.0, 147.3, 139.5, 138.9, 135.6, 132.8, 130.7, 130.5, 130.0, 129.5, 129.4, 129.1, 128.0, 127.1, 116.2, 114.9, 106.3, 67.8, 64.0, 54.7, 12.8, 12.7. HRMS calculated for C₂₇H₂₉N₄O₂ (M+H)+ m/z: 441.2291, measured 441.2291.

B. Administration

To induce or promote wound healing, reduce wound duration, or otherwise aid or augment the wound healing process, one would generally contact a wound with the pyrvinium compound, or salts or analogs thereof. The terms “administered,” “contacted,” “provided” and “exposed,” when applied to a wound or tissue, are used herein to describe the process by which a therapeutic agent is brought into contact or placed in direct juxtaposition with the target. To achieve therapeutic benefit as defined above, the therapeutic agent is delivered to a target in an effective amount.

Pyrvinium or a salt or an analog thereof as a therapeutic agent may be provided to a subject more than once and at intervals ranging from minutes to weeks. One would generally ensure that a significant period of time did not expire between the time of each delivery, such that the agent would still be able to exert an continuous effect on the target. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations.

Administration of pyrvinium or a salt or an analog thereof to a subject may be by any method know in the art for delivery of a therapeutic agent to a subject. For example, such methods may include, but are not limited to, oral, nasal, intramuscular, or intraperitoneal administration. Methods of administration are disclosed in detail elsewhere in this application.

### III. WOUND HEALING

Wound healing, or wound repair, is an intricate process in which the skin (or another organ-tissue) repairs itself after injury. In normal skin, the epidermis (outermost layer) and dermis (inner or deeper layer) exists in a steady-state equilibrium, forming a protective barrier against the external environment. Once the protective barrier is broken, the normal (physiologic) process of wound healing is immediately set in motion. The classic model of wound healing is divided into three or four sequential yet overlapping phases: (1) hemostasis, (2) inflammatory, (3) proliferative and (4) remodeling. Upon injury to the skin, a set of complex biochemical events takes place in a closely orchestrated cascade to repair the damage. Within minutes post-injury, platelets (thrombocytes) aggregate at the injury site to form a fibrin clot. This clot acts to control active bleeding (hemostasis).

In the inflammatory phase, bacteria and debris are phagocytosed and removed, and factors are released that cause the migration and division of cells involved in the proliferative phase.

The proliferative phase is characterized by angiogenesis, collagen deposition, granulation tissue formation, epithelialization, and wound contraction. In angiogenesis, new blood vessels are formed by vascular endothelial cells. In fibroplasia and granulation tissue formation, fibroblasts grow and form a new, provisional extracellular matrix (ECM) by excreting collagen and fibronectin. Concurrently, re-epithelialization of the epidermis occurs, in which epithelial cells proliferate and ‘crawl’ atop the wound bed, providing cover for the new tissue.

In contraction, the wound is made smaller by the action of myofibroblasts, which establish a grip on the wound edges and contract themselves using a mechanism similar to that in smooth muscle cells. When the cells' roles are close to complete, unneeded cells undergo apoptosis.

In the maturation and remodeling phase, collagen is remodeled and realigned along tension lines and cells that are no longer needed are removed by apoptosis. However, this process is not only complex but fragile, and susceptible to interruption or failure leading to the formation of chronic non-healing wounds. Factors which may contribute to this include diabetes, venous or arterial disease, old age, and infection. The phases of wound healing normally progress in a predictable, timely manner; if they do not, healing may progress inappropriately to either a chronic wound such as a venous ulcer or pathological scarring such as a keloid scar.

Treatment of wounds depends on how severe the wound is, its location, and whether other areas are affected. If another condition is causing problems with wound healing, it is important to treat or control this problem. A caregiver may prescribe antibiotics to fight infection, either orally, i.v., or applied directly on the wound area. Palliative care such as for pain, swelling and fever are often prescribed. Wound care is essential as well and includes cleansing, debridement and wound dressing. Dressings are particularly important to protect the wound from further injury and infection. These may also help give pressure to decrease swelling. Dressings may be in the form of bandages, films, or foams. They may contain certain substances that may help promote faster healing. Sometimes, skin taken from another part of the body may be used to close a large wound. The skin may also be man-made, which contains special cells needed to repair damaged tissues. Additional treatments include hyperbaric oxygen therapy (HBO), negative pressure therapy (also called vacuum-assisted closure or “VAC”), or creams, ointments, or medicines with special solutions which help in wound healing may be applied to the wound.

### IV. COMBINED THERAPIES

In the context of the present invention, it is contemplated that the pyrvinium compound, or salts or analogs thereof may be used in combination with a second therapeutic agent to more effectively treat wounds. Additional therapeutic agents contemplated for use in combination with the pyrvinium compound, or salts or analogs thereof include, but are not limited to other wound healing agents, protective agents, and scar reducing agents and the like. Specific examples include corticosteroids, cytotoxic drugs, antibiotics, antiseptics, nicotine, anti-platelet drugs, NSAIDS, colchicines, anti-coagulants, vasoconstricting drugs and immunosuppressives, as well as HBO and VAC methods, discussed above.

To aid in the wound healing process, using the methods and compositions of the present invention, one would generally contact a cell with a pyrvinium compound, or salts or analogs thereof in combination with a second agent. These compositions would be provided in a combined amount effective to exert a combined effect on the damaged tissue. This process may involve contacting the cells with pyrvinium, or salts or analogs thereof in combination with a second therapeutic agent or factor(s) at the same time. This may be achieved by contacting the cell with a single composition or pharmacological formulation that includes both agents, or by contacting the cell with two distinct compositions or formulations, at the same time, wherein one composition includes the pyrvinium or derivatives thereof and the other includes the second agent.

Alternatively, treatment with pyrvinium, or salts or analogs thereof may precede or follow the additional agent treatment by intervals ranging from minutes to weeks. In embodiments where the second agent is applied separately to the target, one would generally ensure that a significant period of time did not expire between the time of each delivery, such that the agent would still be able to exert an advantageously combined effect on the target. In such instances, it is contemplated that one would contact the target with both modalities within about 12-24 hr of each other and, more preferably, within about 6-12 hr of each other. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations.

It also is conceivable that more than one administration of either pyrvinium or salts, or analogs thereof in combination with a second therapeutic agent will be desired. Various combinations may be employed, where pyrvinium or salts or analogs thereof is “A” and the second therapeutic agent is “B”, as exemplified below:

A/B/A B/A/B B/B/A A/A/B B/A/A A/B/B B/B/B/A
B/B/A/B A/A/B/B A/B/A/B A/B/B/A B/B/A/A B/A/B/A
B/A/A/B B/B/B/A A/A/A/B B/A/A/A A/B/A/A A/A/B/A
A/B/B/B B/A/B/B B/B/A/B

Other combinations are contemplated.

In the present invention, a number of drugs or agents may prove particularly useful when combined with pyrvinium or salts or analogs thereof. Such agents/drugs include corticosteroids, NSAIDs or any other anti-inflammatory, a cytotoxic drug, an antibiotic, antimicrobial, antifungal or antiseptic, nicotine, an anti-platelet drug, colchicine, anti-coagulants, vasoconstricting drugs or immunosuppressives.

### V. FORMULATIONS AND ROUTES FOR ADMINISTRATION

Where clinical applications are contemplated, it will be necessary to prepare pharmaceutical compositions of pyrvinium or salts or analogs thereof, or any additional therapeutic agent disclosed herein in a form appropriate for the intended application. Generally, this will entail preparing compositions that are essentially free of pyrogens, as well as other impurities that could be harmful to humans or animals.

One will generally desire to employ appropriate salts and buffers to allow for proper administration. Aqueous compositions of the present invention in an effective amount may be dissolved or dispersed in a pharmaceutically acceptable carrier or aqueous medium. Such compositions also are referred to as inocula. The phrase “pharmaceutically or pharmacologically acceptable” refers to molecular entities and compositions that do not produce adverse, allergic, or other untoward reactions when administered to an animal or a human. As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the agents of the present invention, its use in therapeutic compositions is contemplated. Supplementary active ingredients also can be incorporated into the compositions.

The composition(s) of the present invention may be delivered orally, nasally, intramuscularly, or intraperitoneally, but in particular the invention is designed for topical or mucosal application. In some embodiments, local or regional delivery of pyrvinium or salts or analogs thereof alone, or in combination with a second therapeutic agent, to a wound are contemplated. Other examples of delivery of the compounds of the present invention that may be employed include intra-arterial, intracavity, intravesical, intrathecal, intrapleural, and intraperitoneal routes. Systemic delivery may be appropriate in certain circumstances.

The active compositions of the present invention may include classic pharmaceutical preparations. Solutions of the active compounds as free base or pharmacologically acceptable salts can be prepared in water suitably mixed with a surfactant, such as hydroxypropylcellulose. Dispersions also can be prepared in glycerol, liquid polyethylene glycols, and mixtures thereof and in oils. Under ordinary conditions of storage and use, these preparations contain a preservative to prevent the growth of microorganisms. The therapeutic compositions of the present invention may be administered in the form of injectable compositions either as liquid solutions or suspensions; solid forms suitable for solution in, or suspension in, liquid prior to injection may also be prepared. These preparations also may be emulsified.

A typical composition for such purpose comprises a pharmaceutically acceptable carrier. For instance, the composition may contain 10 mg, 25 mg, 50 mg or up to about 100 mg of human serum albumin per milliliter of phosphate buffered saline. Other pharmaceutically acceptable carriers include aqueous solutions, non-toxic excipients, including salts, preservatives, buffers and the like. Examples of non-aqueous solvents are propylene glycol, polyethylene glycol, vegetable oil and injectable organic esters such as ethyloleate. Aqueous carriers include water, alcoholic/aqueous solutions, saline solutions, parenteral vehicles such as sodium chloride, Ringer's dextrose, etc. Intravenous vehicles include fluid and nutrient replenishers. Preservatives include antimicrobial agents, anti-oxidants, chelating agents and inert gases. The pH, exact concentration of the various components, and the pharmaceutical composition are adjusted according to well known parameters. Suitable excipients for formulation with pyrvinium or salts or analogs thereof include croscarmellose sodium, hydroxypropyl methylcellulose, iron oxides synthetic), magnesium stearate, microcrystalline cellulose, polyethylene glycol 400, polysorbate 80, povidone, silicon dioxide, titanium dioxide, and water (purified).

Additional formulations are suitable for oral administration. Oral formulations include such typical excipients as, for example, pharmaceutical grades of mannitol, lactose, starch, magnesium stearate, sodium saccharine, cellulose, magnesium carbonate and the like. The compositions take the form of solutions, suspensions, tablets, pills, capsules, sustained release formulations or powders. When the route is topical, the form may be a cream, ointment, salve or spray.

An effective amount of the therapeutic agent(s) of the present invention is determined based on the intended goal, for example (i) inhibition of tumor cell proliferation or (ii) elimination of tumor cells. The term “unit dose” refers to physically discrete units suitable for use in a subject, each unit containing a predetermined-quantity of the therapeutic composition calculated to produce the desired responses, discussed above, in association with its administration, i.e., the appropriate route and treatment regimen. The quantity to be administered, both according to number of treatments and unit dose, depends on the subject to be treated, the state of the subject and the protection desired. Precise amounts of the therapeutic composition also depend on the judgment of the practitioner and are peculiar to each individual.

### VI. THERAPEUTICALLY EFFECTIVE AMOUNTS OF PYRVINIUM COMPOSITIONS

A therapeutically effective amount of pyrvinium, or salts, or analogs thereof alone, or in combination with a second therapeutic agent such as an anticancer agent as a treatment varies depending upon the host treated and the particular mode of administration. In one embodiment of the invention the dose range of the pyrvinium or salts or analogs thereof alone, or in combination with a second agent used will be about 0.5 mg/kg body weight to about 500 mg/kg body weight. The term “body weight” is applicable when an animal is being treated. When isolated cells are being treated, “body weight” as used herein should read to mean “total cell weight”. The term “total weight may be used to apply to both isolated cell and animal treatment. All concentrations and treatment levels are expressed as “body weight” or simply “kg” in this application are also considered to cover the analogous “total cell weight” and “total weight” concentrations. However, those of skill will recognize the utility of a variety of dosage range, for example, 1 mg/kg body weight to 450 mg/kg body weight, 2 mg/kg body weight to 400 mg/kg body weight, 3 mg/kg body weight to 350 mg/kg body weight, 4 mg/kg body weight to 300 mg/kg body weight, 5 mg/kg body weight to 250 mg/kg body weight, 6 mg/kg body weight to 200 mg/kg body weight, 7 mg/kg body weight to 150 mg/kg body weight, 8 mg/kg body weight to 100 mg/kg body weight, or 9 mg/kg body weight to 50 mg/kg body weight. Further, those of skill will recognize that a variety of different dosage levels will be of use, for example, 1 mg/kg, 2 mg/kg, 3 mg/kg, 4 mg/kg, 5 mg/kg, 7.5 mg/kg, 10 mg/kg, 12.5 mg/kg, 15 mg/kg, 17.5 mg/kg, 20 mg/kg, 25 mg/kg, 30 mg/kg, 35 mg/kg, 40 mg/kg, 45 mg/kg, 50 mg/kg, 60 mg/kg, 70 mg/kg, 80 mg/kg, 90 mg/kg, 100 mg/kg, 120 mg/kg, 140 mg/kg, 150 mg/kg, 160 mg/kg, 180 mg/kg, 200 mg/kg, 225 mg/kg, 250 mg/kg, 275 mg/kg, 300 mg/kg, 325 mg/kg, 350 mg/kg, 375 mg/kg, 400 mg/kg, 450 mg/kg, 500 mg/kg, 550 mg/kg, 600 mg/kg, 700 mg/kg, 750 mg/kg, 800 mg/kg, 900 mg/kg, 1000 mg/kg, 1250 mg/kg, 1500 mg/kg, 1750 mg/kg, 2000 mg/kg, 2500 mg/kg, and/or 3000 mg/kg. Of course, all of these dosages are exemplary, and any dosage in-between these points is also expected to be of use in the invention. Any of the above dosage ranges or dosage levels may be employed for pyrvinium, or salts or analogs thereof in combination with a second therapeutic agent.

“Therapeutically effective amounts” are those amounts effective to produce beneficial results, particularly with respect to wound healing, in the recipient animal or patient. Such amounts may be initially determined by reviewing the published literature, by conducting in vitro tests or by conducting metabolic studies in healthy experimental animals. Before use in a clinical setting, it may be beneficial to conduct confirmatory studies in an animal model, preferably a widely accepted animal model of the particular disease to be treated. Preferred animal models for use in certain embodiments are rodent models, which are preferred because they are economical to use and, particularly, because the results gained are widely accepted as predictive of clinical value.

As is well known in the art, a specific dose level of active compounds such as pyrvinium or salts or analogs thereof alone, or in combination with a second therapeutic agent, for any particular patient depends upon a variety of factors including the activity of the specific compound employed, the age, body weight, general health, sex, diet, time of administration, route of administration, rate of excretion, drug combination, and the severity of the particular disease undergoing therapy. The person responsible for administration will determine the appropriate dose for the individual subject. Moreover, for human administration, preparations should meet sterility, pyrogenicity, general safety and purity standards as required by FDA Office of Biologics standards.

In some embodiments, pyrvinium or salts or analogs thereof alone, or in combination with a second therapeutic agent will be administered. When a second therapeutic agent is administered, as long as the dose of the second therapeutic agent does not exceed previously quoted toxicity levels, the effective amounts of the second therapeutic agents may simply be defined as those amounts effective to reduce the cancer growth when administered to an animal in combination with the pyrvinium or salts or analogs thereof. This may be easily determined by monitoring the animal or patient and measuring those physical and biochemical parameters of health and disease that are indicative of the success of a given treatment. Such methods are routine in animal testing and clinical practice.

In some embodiments of the present invention may be administered, as is typical, in regular cycles. A cycle may involve one dose, after which several days or weeks without treatment ensues for normal tissues to recover from possible side effects. Doses may be given several days in a row, or every other day for several days, followed by a period of rest. If more than one drug is used, the treatment plan will specify how often and exactly when each drug should be given. The number of cycles a person receives may be determined before treatment starts (based on the type, location and severity of the wound) or may be flexible, in order to take into account how quickly the wound is healing. Certain serious side effects may also require doctors to adjust the therapy to allow the patient time to recover.

### VII. DEVICES FOR DELIVERY OF THERAPEUTIC COMPOUNDS

The present invention involves, in some aspects, the provision of devices for delivery of pyrvinium compounds to wounds. In general, it is contemplated that any device or material that is brought into contact with a wound is a suitable vehicle for delivering pyrvinium compounds. The following devices/materials are exemplary in nature and are not meant to be limiting.

A. Wound Dressings

The present invention in one aspect, provides for various wound dressings that incorporate or have applied thereto the pyrvinium compounds of the present invention. Dressings have a number of purposes, depending on the type, severity and position of the wound, although all purposes are focused towards promoting recovery and preventing further harm from the wound. Key purposes of are dressing are to seal the wound and expedite the clotting process, to soak up blood, plasma and other fluids exuded from the wound, to provide pain relieving effect (including a placebo effect), to debride the wound, to protect the wound from infection and mechanical damage, and to promote healing through granulation and epithelialization.

The following list of commercial dressings includes those that may be employed in accordance with the present invention: Acticoat, Acticoat 7, Actisorb Silver 220, Algisite M, Allevyn, Allevyn Adhesive, Allevyn Cavity, Allevyn Compression, Allevyn Heel, Allevyn Sacrum, Allevyn cavity wound dressing, Aquacel, Aquacel AG, Aquacel ribbon, Bactigras, Biatain Adhesive, Bioclusive, Biofilm, Blenderm, Blue line webbing, Bordered Granuflex, Calaband, Carbonet, Cavi-care, Cellacast Xtra, Cellamin, Cellona Xtra, Cellona elastic, Chlorhexitulle, Cica-Care, Cliniflex odour control dressing, Clinisorb odour control dressing, Coban, Coltapaste, Comfeel Plus, Comfeel Plus pressure relieving dressing, Comfeel Plus transparent dressing, Comfeel Plus ulcer dressing, Comfeel seasorb dressing, Comfeel ulcer dressing, Contreet Non-Adhesive, Crevic, Cutinova Hydro, Cutinova Hydro Border, Debrisan absorbent pad, Debrisan beads, Debrisan paste, Delta-Cast Black Label, Delta-Cast conformable, Delta-Lite S, Duoderm extra thin, Durapore, Elastocrepe, Elset/Elset ‘S’, Flamazine, Fucidin Intertulle, Geliperm granulated gel, Geliperm sheet, Granuflex (Improved formulation), Granuflex extra thin, Granugel, Gypsona, Gypsona S, Hypafix, Icthaband, Icthopaste, Inadine, Intrasite Gel, Iodoflex, Iodosorb, Iodosorb ointment, Jelonet, K-Band, K-Lite, K-PLUS, Kaltocarb, Kaltostat, Kaltostat Fortex, Kaltostat cavity dressing, LarvE (Sterile Maggots), Lestreflex, Lyofoam, Lyofoam ‘A’, Lyofoam C, Mefix, Melolin, Mepiform, Mepilex, Mepilex AG, Mepilex Border, Mepilex Border Lite, Mepilex Border Sacrum, Mepilex Heel, Mepilex Lite, Mepilex Transfer, Mepitac, Mepitel, Mepore, Mepore Pro, Mesitran, Mesorb, Metrotop, Microfoam, Micropore, Opsite Flexigrid, Opsite IV 3000, Orthoflex, Oxyzyme, Paratulle, Polymem, Polymem Island & Shapes, Polymem Max, Polymem Silver, ProGuide, Profore, Promogran, Quinaband, Release, Scotchcast Plus, Scotchcast Softcast, Serotulle, Setopress, Silastic foam, Silicone N-A, Sofra-Tulle, Sorbsan, Sorbsan Plus, Sorbsan SA, Sorbsan Silver, Sorbsan Silver Plus Self Adhesive, Spenco 2nd Skin, Spyroflex, Spyrosorb, Tarband, Tegaderm, Tegaderm Plus, Tegagel, Tegapore, Tegasorb, Telfa, Tensopress, Tielle, Tielle Lite, Tielle Plus, Tielle Plus Borderless, Transpore, Unitulle, Veinoplast, Veinopress, Versiva, Vigilon, Viscopaste PB7, Xelma, and Zincaband.

A typical (sterile) dressing is one made of a film, foam, semi-solid gel, pad, gauze, or fabric. More particularly, sterile dressings are made of silicone, a fibrin/fibrinogen matrix, polyacrylamide, PTFE, PGA, PLA, PLGA, a polycaprolactone or a hyaluronic acid, although the number and type of materials useful in making dressings is quite large. Dressing may further be described as compression dressings, adherent dressing and non-adherent dressings.

Dressings may advantageously include other materials—active or inert. Such materials include gelatin, silver, cellulose, an alginate, collagen, a hydrocolloid, a hydrogel, a skin substitute, a wound filler, a growth factor, an antibody, a protease, a protease inhibitor, an antibacterial peptide, an adhesive peptide, a hemostatic agent, living cells, honey, nitric oxide, a corticosteroid, a cytotoxic drug, an antibiotic, an antimicrobial, an antifungal, an antiseptic, nicotine, an anti-platelet drug, an NSAID, colchicine, an anti-coagulant, a vasoconstricting drug or an immunosuppressive.

Wound dressings may also be part of a larger device, such as one that permits fixation of the dressing to a wound, such as an adhesive or a bandage. Dressings/devices may also include other features such as a lubricant, to avoid adhesion of the dressing to the wound, an absorber to remove seepage from the wound, padding to protect the wound, a sponge for absorbance or protection, a wound veil, an odor control agent, and/or a cover.

The pyrvinium agent, or any other agent, may be applied to a dressing, or disposed in a dressing, by virtue of its introduction into or onto the dressing in a liquid, a salve, an ointment, a gel or a powder. Alternatively, the pyrvinium agent or other agent may be added to a discrete element of a dressing (a sheet or film) that is included in the dressing during its manufacture.

Devices may also include a port, such as one providing operable connection between said sterile dressing and a tube, as well as a cover providing an airtight seal to or around a wound surface. Such embodiments are particularly useful in negative pressure wound therapy methods and devices.

B. Sutures

A surgical suture is a medical device used to hold body tissues together after an injury or surgery. It generally a length of thread, and it attached to a needle. A number of different shapes, sizes, and thread materials have been developed over time. The present invention envisions the coating or impregnating of sutures with pyrvinium compounds.

The first synthetic absorbable was based on polyvinyl alcohol in 1931. Polyesters were developed in the 1950s, and later the process of radiation sterilization was established for catgut and polyester. Polyglycolic acid was discovered in the 1960s and implemented in the 1970s. Today, most sutures are made of synthetic polymer fibers, including the absorbables polyglycolic acid, polylactic acid, and polydioxanone as well as the non-absorbables nylon and polypropylene. More recently, coated sutures with antimicrobial substances to reduce the chances of wound infection have been developed. Sutures come in very specific sizes and may be either absorbable (naturally biodegradable in the body) or non-absorbable. Sutures must be strong enough to hold tissue securely but flexible enough to be knotted. They must be hypoallergenic and avoid the “wick effect” that would allow fluids and thus infection to penetrate the body along the suture tract.

All sutures are classified as either absorbable or non-absorbable depending on whether the body will naturally degrade and absorb the suture material over time. Absorbable suture materials include the original catgut as well as the newer synthetics polyglycolic acid (Biovek), polylactic acid, polydioxanone, and caprolactone. They are broken down by various processes including hydrolysis (polyglycolic acid) and proteolytic enzymatic degradation. Depending on the material, the process can be from ten days to eight weeks. They are used in patients who cannot return for suture removal, or in internal body tissues. In both cases, they will hold the body tissues together long enough to allow healing, but will disintegrate so that they do not leave foreign material or require further procedures. Occasionally, absorbable sutures can cause inflammation and be rejected by the body rather than absorbed.

Non-absorbable sutures are made of special silk or the synthetics polypropylene, polyester or nylon. Stainless steel wires are commonly used in orthopedic surgery and for sternal closure in cardiac surgery. These may or may not have coatings to enhance their performance characteristics. Non-absorbable sutures are used either on skin wound closure, where the sutures can be removed after a few weeks, or in stressful internal environments where absorbable sutures will not suffice. Examples include the heart (with its constant pressure and movement) or the bladder (with adverse chemical conditions). Non-absorbable sutures often cause less scarring because they provoke less immune response, and thus are used where cosmetic outcome is important. They must be removed after a certain time, or left permanently.

In recent years, topical cyanoacrylate adhesives (liquid stitches”) have been used in combination with, or as an alternative to, sutures in wound closure. The adhesive remains liquid until exposed to water or water-containing substances/tissue, after which it cures (polymerizes) and forms a flexible film that bonds to the underlying surface. The tissue adhesive has been shown to act as a barrier to microbial penetration as long as the adhesive film remains intact. Limitations of tissue adhesives include contraindications to use near the eyes and a mild learning curve on correct usage.

Cyanoacrylate is the generic name for cyanoacrylate based fast-acting glues such as methyl-2-cyanoacrylate, ethyl-2-cyanoacrylate (commonly sold under trade names like Superglue™ and Krazy Glue™) and n-butyl-cyanoacrylate. Skin glues like Indermil® and Histoacryl® were the first medical grade tissue adhesives to be used, and these are composed of n-butyl cyanoacrylate. These worked well but had the disadvantage of having to be stored in the refrigerator, were exothermic so they stung the patient, and the bond was brittle. Nowadays, the longer chain polymer, 2-octyl cyanoacrylate, is the preferred medical grade glue. It is available under various trade names, such as LiquiBand®, SurgiSeal®, FloraSeal®, and Dermabond®. These have the advantages of being more flexible, making a stronger bond, and being easier to use. The longer side chain types, for example octyl and butyl forms, also reduce tissue reaction.

C. Negative Pressure Wound Therapy

Negative pressure wound therapy (NPWT), also known as topical negative pressure, sub-atmospheric pressure dressings or vacuum sealing technique, is a therapeutic technique used to promote healing in acute or chronic wounds, fight infection and enhance healing of burns. A vacuum source is used to create sub-atmospheric pressure in the local wound environment. The wound is sealed to prevent dehiscence with a gauze or foam filler dressing, and a drape and a vacuum source applies negative pressure to the wound bed with a tube threaded through the dressing. The vacuum may be applied continuously or intermittently, depending on the type of wound being treated and the clinical objectives. Intermittent removal of used instillation fluid supports the cleaning and drainage of the wound bed and the removal of infectious material.

NPWT has multiple forms which mainly differ in the type of dressing used to transfer NPWT to the wound surface, and include both gauze and foam. Gauze has been found to effect less tissue ingrowth than foam. The dressing type depends on the type of wound, clinical objectives and patient. For pain sensitive patients with shallow or irregular wounds, wounds with undermining or explored tracts or tunnels, and for facilitating wound healing, gauze may be a better choice for the wound bed, while foam may be cut easily to fit a patient's wound that has a regular contour and perform better when aggressive granulation formation and wound contraction is the desired goal. The technique is often used with chronic wounds or wounds that are expected to present difficulties while healing (such as those associated with diabetes or when the veins and arteries are unable to provide or remove blood adequately).

### VIII. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Pyrvinium Inhibits Wnt Signaling

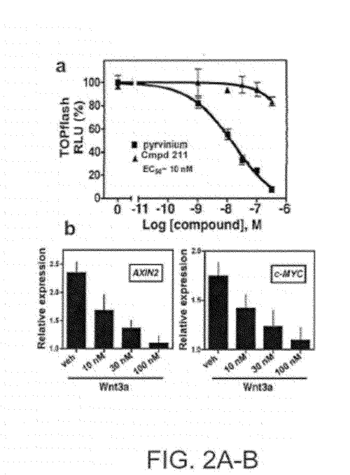

The inventors developed a biochemical assay using Xenopus laevis egg extract that recapitulates Axin and β-catenin turnover in response to addition of recombinant Wnt co-receptor (LRP6) (Cselenyi et al., 2008). In Xenopus egg extract, β-catenin is robustly degraded and Axin is stable. In the presence of LRP6, however, β-catenin degradation is inhibited and Axin degradation is stimulated. Using this system (with β-catenin fused to firefly luciferase and Axin fused to Renilla luciferase as reporters for protein levels), they performed a high-throughput screen to identify small molecules that reverse the effects of recombinant LRP6. The inventors identified an FDA-approved antihelminthic compound (pyrvinium) that promotes β-catenin degradation and inhibits Axin degradation in Xenopus extract. Using a TOPflash reporter cell line (293HEK STF) in which luciferase is under the control of the TCF/Lef1 promoter, the inventors found that pyrvinium inhibits Wnt signaling with an IC₅₀ of ˜10 nM in contrast to a control compound with a similar structure (Cmpd 211; FIG. 2A). Inhibition of Wnt signaling was further confirmed by real-time RT-PCR of endogenous Wnt target genes, Axin2 and c-MYC (FIG. 2B).

Mechanism of Pyrvinium Action.

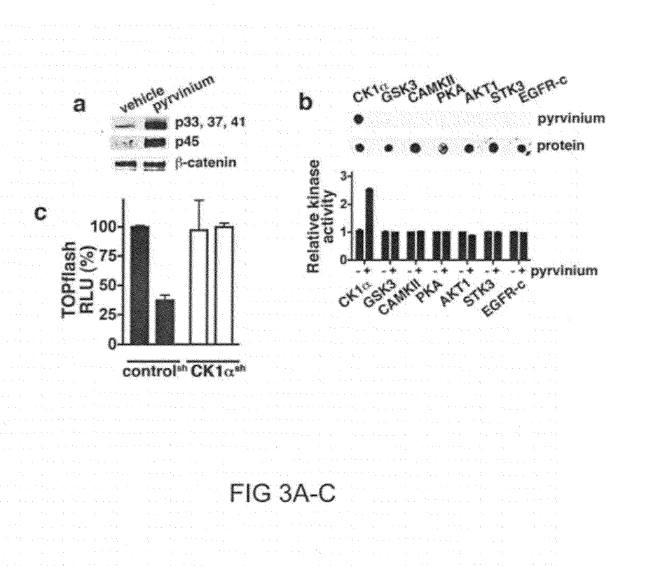

To test whether pyrvinium acts directly on the β-catenin degradation complex, the inventors assembled an in vitro reaction consisting of purified GSK3, CK1α, Axin, and β-catenin and tested the effect of pyrvinum on β-catenin phosphorylation, a prerequisite for its degradation. Addition of pyrvinium to this system resulted in a dramatic increase in β-catenin phosphorylation, including sites specific for GSK3 and CK1α (FIG. 3A). Pyrvinium enhanced phosphorylation of Tau by CK1α, but had no observable effect on GSK3 activity (data not shown). To demonstrate specificity for CK1α, the inventors tested the capacity of pyrvinium to bind and activate representative kinases from major branches of the kinase superfamily. Of the kinases tested, they observed pyrvinium binding and activation of CK1α alone (FIG. 3B). If pyrvinium inhibits Wnt signaling via activation of CK1α, loss of CK1α should block the effects of pyrvinium on Wnt signaling. The inventors used a cell line with reduced CK1α levels due to inducible expression of shRNA against CK1α. They found that, in contrast to control cells, pyrvinium failed to inhibit TOPflash activity in shRNA-CK1α cells (FIG. 3C). These results indicate that the effects of pyrvinium on Wnt signaling are mediated by its activation of CK1α.

Wnt Inhibition by Pyrvinium Promotes Advanced, Vascularized Granulation Tissue in a Murine Model.

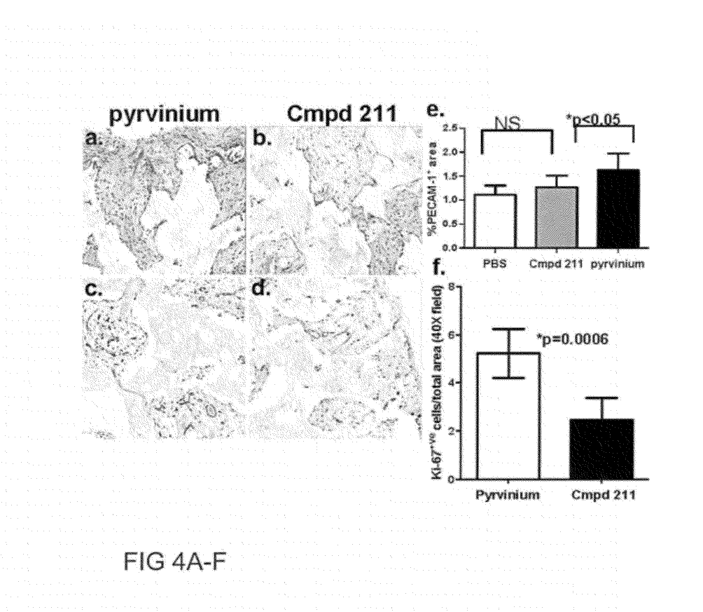

The deposition of granulation tissue after wounding is a critical step in tissue regeneration (Inoue et al., 1998). To determine the effect of Wnt inhibition in promoting both the quantity and quality of granulation tissue deposition (Davidson et al., 1985; Li et al., 2005), the inventors implanted polyvinyl alcohol (PVA) sponge discs (isolate granulation tissue from epithelial reformation) subcutaneously beneath the ventral panniculus carnosus in adult mice. Each mouse (n=6) was implanted with four sponges and sacrificed on day 14. Each sponge was injected on day (d) 2, 4, 6 and 8, 10, and 12 with 50 μl of the following agents reconstituted in PBS/1% albumin: Sponge 1 and 2 received 200 nM pyrvinium, sponge 3 received 200 nM Cmpd 211, and sponge 4 received vehicle alone. A range of pyrivnium doses (25-300 nM) was tested to determine the optimal dose that enhances proliferation of mesenchymal stem cells (MSCs) in vitro (data not shown). FIGS. 4A-F is a representative H&E stained section obtained from the same mouse of a sponge receiving pyrvinium or Cmpd 211. Granulation tissue from sponges treated with Cmpd 211 or vehicle (data not shown) exhibited loose, disorganized architecture. In contrast, granulation tissue from pyrvinium-treated sponges exhibited more cellularity and better tissue organization. Vascular density of the granulation tissue was greater in pyrvinium-treated sponges compared to Cmpd 211-treated sponges when histologic sections were assessed by anti-PECAM-1 staining (marks endothelial cells) (FIGS. 4C-E). Cellular proliferation of granulation tissue was assessed by immunostaining for the nuclear protein Ki-67, a marker for proliferation (FIG. 4F). Pyrvinium treatment of PVA sponges resulted in approximately 2.5-fold increase in cell proliferation of the resultant granulation tissue (FIG. 4F).

A Single Administration of Pyrvinium Results in Improved Cardiac Remodeling in a Murine Acute Myocardial Infarct Model.

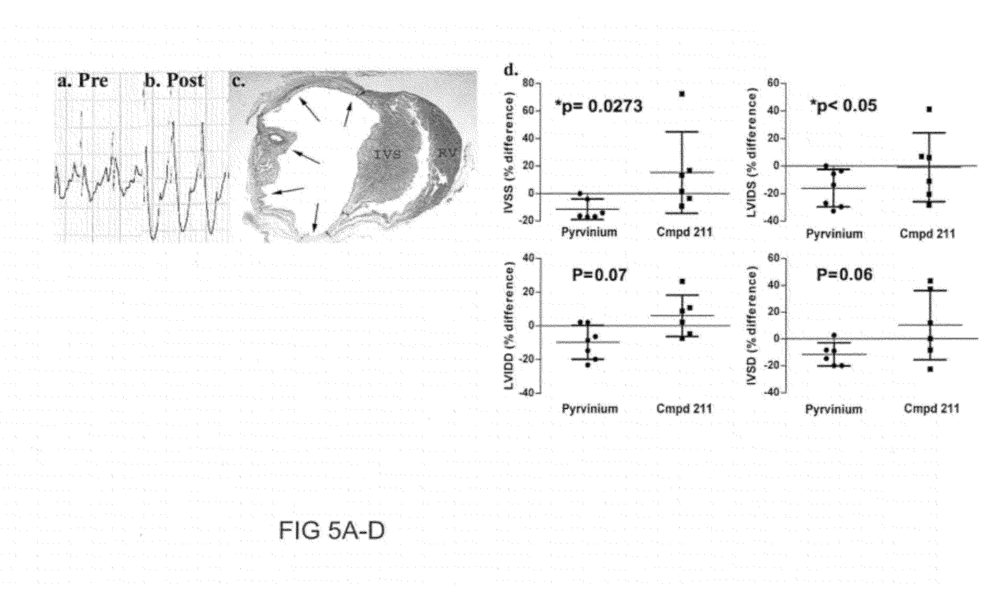

Myocardial infarcts were induced in male C57Bl/6 mice by coronary ligation (FIG. 5) as previously described (Alfaro et al., 2008). 30 min post-ligation, the inventors injected 200 nM of pyrvinium or Cmpd 211 at the junction of viable and infarcted tissue. A large number (15) of mice treated with intracardiac pyrvinium experienced lethal toxicity and died within the first 24 hours. Animals that survived beyond the first 48 hours demonstrated similar activity and growth pattern as the control animals. Toxicity associated with pyrvinium was anticipated as IV or IP injection of pyrvinium at levels high enough to achieve Wnt-inhibitory plasma levels resulted in death within 24-48 hours in most mice.

Left coronary artery ligation produced infarcts in the anterolateral wall of the LV (FIG. 5). Ventricular remodeling and cardiac functional parameters will be assessed by echo. All four (LVIDD, LVIDS, IVSS, and IVSD) dimensional parameters (as analyzed by percentage difference between days 7 and 30 echo measurements) were smaller in the pyrvinium-treated recipients compared to those receiving Cmpd 211, providing strong support for post-injury Wnt inhibition as a means to prevent adverse chamber remodeling (FIG. 5). In particular, the percent difference between LVIDD, LVIDS were statistically significant (p<0.05). The data set available thus far remains small and additional numbers are necessary. The average difference in fractional shortening of pyrvinium-treated animals between days 7 and 30 was 22.3±5.2 vs 15.4±8.3 (p<0.05). Comparison of infarct size between the two cohorts also did not reflect any statistically significant differences (data not shown). This observation, along with the absence of functional or anatomic difference between the two cohorts at day 7, provides greater evidence that pyrvinium did not acutely affect the extent of the infarct. Because the inventors were limited to a single administration of pyrvinium after injury, they are unable to confidently assess the effect of Wnt inhibition on cardiac function/infarct size.

Wnt Inhibition by Pyrvinium Increases Cardiomyoctye Mitosis in the Postmitotic Myocardium.

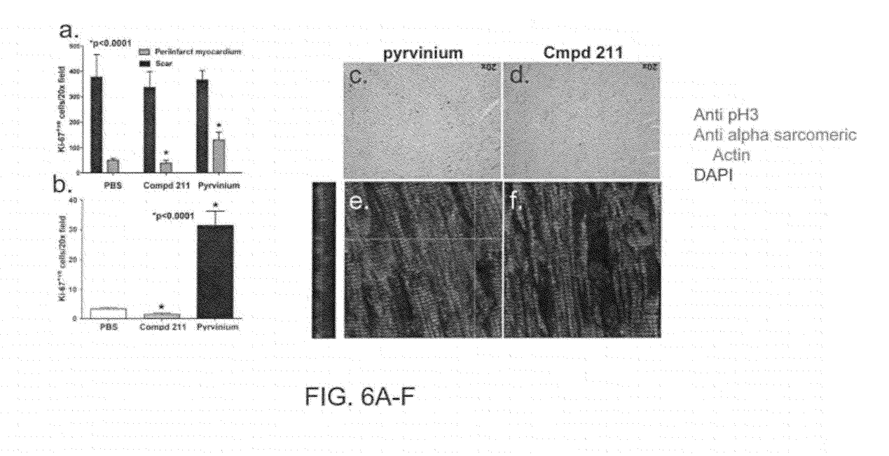

Mammalian cardiomyocytes irreversibly withdraw from the cell cycle soon after birth and undergo terminal differentiation. DNA synthesis, karyokinesis, and cytokinesis do not occur (or occur at very low levels) in adult murine cardiomyocytes 3 weeks after birth (Beinlich and Morgan, 1993; Simpson, 1989). Therefore, cardiac injury causes permanent myocardial loss and cardiac dysfunction. The inventors tested the hypothesis that favorable remodeling mediated by pyrvinium may be through inducing mitosis of adult cardiomyoctyes. Proliferation was assessed by immunostaining for Ki-67. While the numbers of Ki-67-positive cells were similar in the scar of both control Cmpd 211- and pyrvinium-treated animals, in the peri-infarct and, strikingly, in the remote myocardium, the numbers of Ki-67-positive cells were significantly higher in the pyrvinium-treated hearts (FIGS. 6A-D). Phosphorylation of histone 3 (pH3) on Ser10 is an established cellular marker for chromosome condensation during mitotic prophase (van Amerongen and Engel, 2008). The inventors immunostained for pH3 and performed confocal microscopy to assess the effect of Wnt inhibition on the mitotic status of cardiomyocytes. pH3+ (red) cells exhibited a differentiated phenotype as indicated by striations and expression of α-sarcomeric actin (green). Reconstruction of optical sections enabled us to assign pH3-positive nuclei unequivocally to cardiomyocytes (side panel). Importantly, pyrvinium did not induce myocyte proliferation in sham-operated animals that received a single intramyocardial injection into LV apex (data not shown).

Wnt Analog has CK1α Activating Activity.

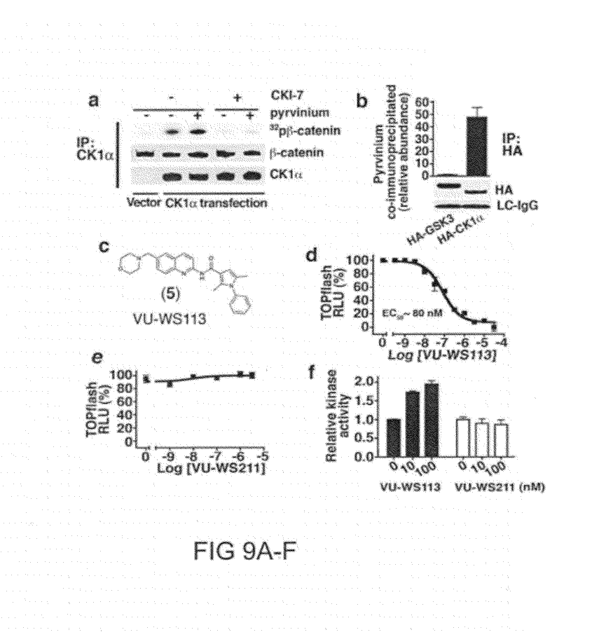

If Wnt pathway inhibition by pyrvinium were mediated by CK1α activation, a structural analog of pyrvinium that has retained the capacity to inhibit Wnt signaling (VU-WS113) should also activate CK1α. Indeed, we found this to be the case (FIGS. 9C-F). In contrast, the Cmpd 211 derivative that does not inhibit Wnt signaling failed to activate CK1α (FIGS. 9C-F).

Summary.

These data show that pyrvinium, a potent and specific small molecule inhibitor of canonical Wnt signaling, promoted better organized and vascularized granulation tissue in vivo compared to control. The inventors show that mice treated with peri-infarct intramuscular administration of pyrvinium demonstrated significantly more favorable LV remodeling 30 d post-MI compared to a control compound. Remarkably, this effect was observed after only a single dose of intramuscular administration of the Wnt inhibitor following injury. These data further suggest that the cellular basis of Wnt-inhibitor-mediated remodeling is, in part, due to induction of myocyte mitosis. These findings highlight the potential of Wnt inhibition to treat MI and the need for a safe and effective therapeutic Wnt inhibitor to better dissect the effect of Wnt inhibition on cardiac repair and regeneration.

### Example 2

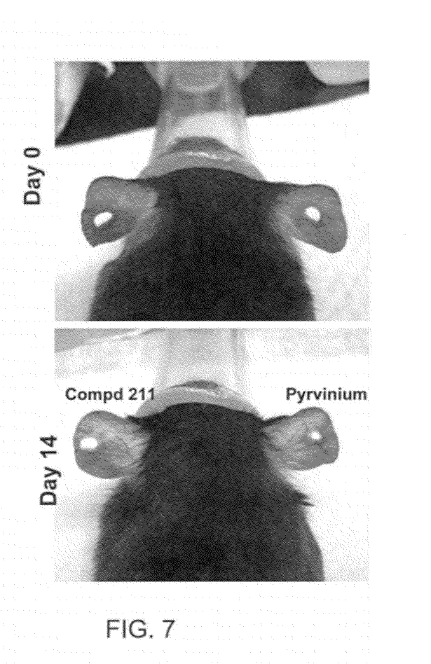

The ears of six month old C57BL/6 mice were wounded by a 2 mm ear punch (n=3). One μM aqueous solution of pyrvinium was applied on right ear and the same concentration dose of control Cmpd 211 was applied to the contralateral left year each day after wound. The wounds were monitored over a period of 1 month and the digital photographs of the healing ears were taken periodically. Within two weeks enhanced closing of the ear hole was noticed in the ears (bottom panel, right ear) that received pyrvinium. The mice were sacrificed at day 30 and histological analysis of the wounded ear was performed. As seen in FIG. 7, the bottom photo, right ear, shows remarkable healing as compared to the left ear at 14 days.

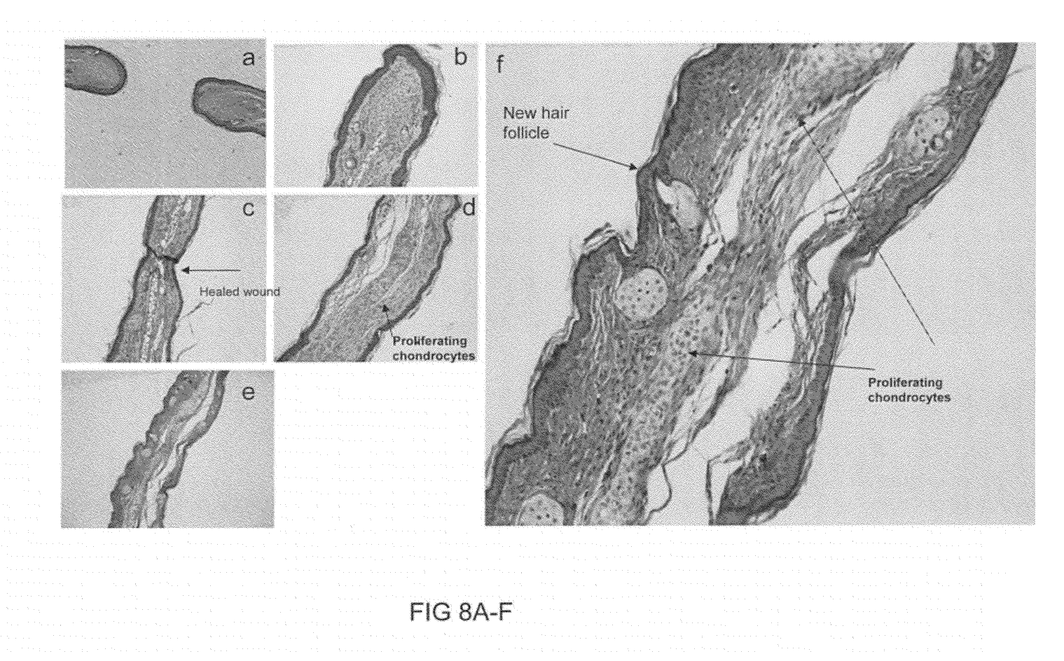

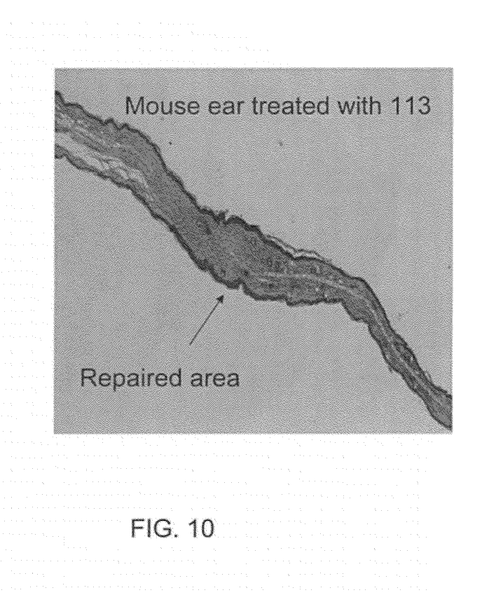

Representative photomicrographs of histologic sections of mice ears treated with control Cmpd 211 are shown in FIGS. 8A-B, while those treated with pyrvinium are shown in FIGS. 8C-F. In FIG. 8A (low magnification—4×) H&E stained slide shows epithelial repair of wounded skin treated with Cmpd 211 at 30 days after injury. The 2 mm hole is evident. In FIG. 8B (high magnification—20×) H&E stained slide shows Cmpd 211-treated ears with no evidence of new hair follicles or cartilage growth near injury. FIGS. 8C-D show H&E stained slide of low (10×) and high (20×) magnifications of pyrvinium-treated ears. The repaired tissue shows evidence of proliferating chondrocytes. FIGS. 8E-F show Trichrome blue staining showing low (FIG. 8E, 10×) and high power (FIG. 8F, 20×) view of pyrvinium repaired ear. Note new hair follicles within repaired area as well as migrating chondrocytes, demonstrating regenerative repair. FIG. 10 shows photomicrograph of a mouse ear treated with Cmpd 113. The injured hole was repaired and tissue proliferation around cartilage was noted.

Pyrvinium Analog C-113 Results in a Dramatic Post-MI Proliferative Response, Notably of Sca1+ Cells.

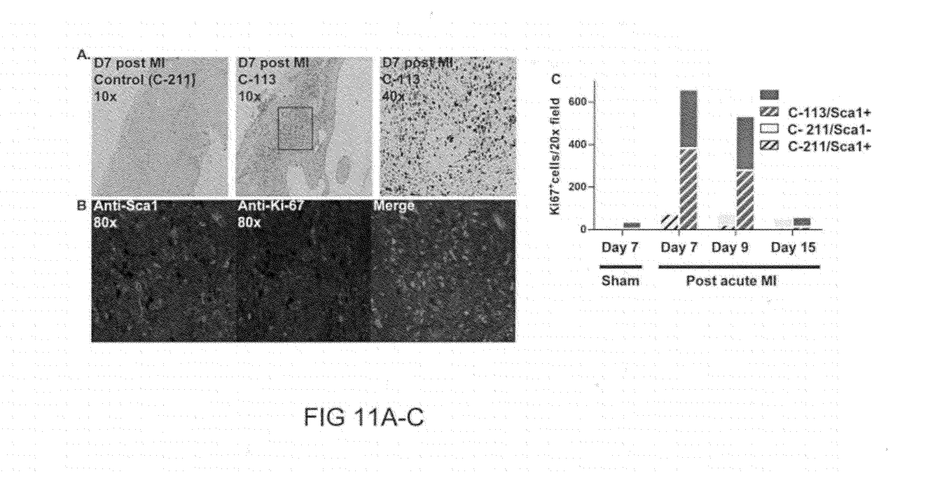

Myocardial infarcts were induced in male C57Bl/6 mice by coronary ligation. A sham-injured cohort underwent all aspects of the procedure except ligation of the coronary artery. 24 hours after injury, mice were injected daily IP with C-113 or Control C-211 (40 μg/kg) through day 6. Mice were sacrificed (n=3 for each time point and each condition) on days 7, 9 and 15 after injury. Hearts were isolated for histologic analysis. C-113 treatment led to a dramatic increase (˜10-13 fold) in left ventricular, peri-infarct Ki67+ cells in mice 7-9 days after MI but not in the LV of sham treated animals (FIG. 11A). The numbers of proliferative cells subsided by day 15 after MI. Notably, at least 50% of the Ki-67+ cells also co-labeled with Sca1+ antibody (FIGS. 11B-C). These data support our hypothesis that inhibition of Wnt signaling in the context of injury results in rapid, peri-infarct cellular proliferation, including marked amplification of CPCs. The finding that Sca1+ CPCs account for >60% of Wnt-inhibitor responsive population also supports our hypothesis that CPCs are uniquely responsive to Wnt modulation.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### IX. References

The following references, to the extent that they provide exemplary procedural or other details supplementary to those set forth herein, are specifically incorporated herein by reference.
U.S. Pat. No. 2,925,417
Arap et al., Cancer Res., 55(6):1351-1354, 1995.
Bakhshi et al., Cell, 41(3):899-906, 1985.
Caldas et al., Nat. Genet., 8(1):27-32, 1994.
Cheng et al., Cancer Res., 54(21):5547-5551, 1994.
Cleary and Sklar, Proc. Natl. Acad. Sci. USA, (21):7439-7443, 1985.
Cleary et al., J. Exp. Med., 164(1):315-320, 1986.
Hollstein et al., Science, 253(5015):49-53, 1991.
Hussussian et al., Nat. Genet., 8(1):15-21, 1994.
Kamb et al., Nat. Genet., 8(1):23-26, 1994.
Kerr et al., Br. J. Cancer, 26(4):239-257, 1972.
Liu et al., Am. J. Gastroenterol., 100:792-796, 2005.
Mori et al., Cancer Res., 54(13):3396-3397, 1994.
Nobri et al., Nature (London), 368:753-756, 1995.
Okamoto et al., Proc. Natl. Acad. Sci. USA, 91(23):11045-11049, 1994.
Orlow et al., Int. J. Oncol., 15(1):17-24, 1994.
Reynolds & Weiss, Science 255: 1707-1710, 1992.
Serrano et al., Nature, 366:704-707, 1993.
Serrano et al., Science, 267(5195):249-252, 1995.
Tsujimoto and Croce, Proc. Natl. Acad. Sci. USA, 83(14):5214-5218, 1986.
Tsujimoto et al., Science, 228(4706):1440-1443, 1985.
Weinberg, Science, 254(5035):1138-1146, 1991.

1. A method of treating a wound in a subject comprising contacting said wound with a composition comprising pyrvinium or a salt or analog thereof.

2. The method of claim 1, wherein said wound is a dermal wound, an epidermal wound, a burn, a laceration or abrasion, an infectious lesion, a surgical site, an ulcer, a puncture, a chronic wound, a scar, a keloid or a blister.

3-13. (canceled)

14. The method of claim 1, wherein pyrvinium or a salt or analog thereof is contacted with said wound in a wound dressing, in a gel, salve or ointment, in a topical spray, a topical liquid or a powder.

15-17. (canceled)

18. The method of claim 1, wherein pyrvinium or a salt or analog thereof is contacted with said wound by injection local or regional to said wound.

19. (canceled)

20. The method of claim 1, further comprising providing to said subject a second wound therapy.

21. The method of claim 20, wherein said second wound therapy is hyperbaric oxygen therapy (HBO), negative pressure therapy (VAC), electrical stimulation, phototherapy or acoustic stimulation.

22. The method of claim 20, wherein said second wound therapy is a corticosteroid, a cytotoxic drug, an antibiotic, an antiseptic, nicotine, an anti-platelet drug, an NSAID, colchicine, an anti-coagulant, a vasoconstricting drug or an immunosuppressive, a growth factor, an antibody, a protease, a protease inhibitor, an antibacterial peptide, an adhesive peptide, a hemostatic agent, living cells, honey, or nitric oxide.

23. The method of claim 1, wherein said subject is a human.

24. The method of claim 1, wherein said subject is a non-human mammal.

25. The method of claim 1, wherein pyrvinium or a salt or analog thereof is contacted with said wound in a suture.

26. The method of claim 1, wherein treating comprises promoting wound tissue hemostasis, promoting wound tissue proliferation, promoting wound tissue contraction, promoting would tissue remodeling, and/or reducing wound tissue scarring in said subject.

27-30. (canceled)

31. A device for the treatment of a wound in a subject comprising:
(a) a composition comprising pyrvinium or a salt or analog thereof;
(b) a sterile dressing into or onto which said pyrvinium, salt or analog is disposed.

32. The device of claim 31, wherein said sterile dressing is a film, foam, semi-solid gel, pad, gauze, fabric.

33. (canceled)

34. The device of claim 31, wherein said sterile dressing further comprises gelatin, silver, cellulose, an alginate, collagen, a hydrocolloid, a hydrogel, a skin substitute, a wound filler, a growth factor, an antibody, a protease, a protease inhibitor, an antibacterial peptide, an adhesive peptide, a hemostatic agent, living cells, honey, or nitric oxide.

35. The device of claim 31, further comprising a substance or element for the fixation of said device to a wound.

36. The device of claim 35, wherein said substance or element is an adhesive or a bandage.

37. (canceled)

38. The device of claim 31, wherein said sterile dressing is a compression dressing or a non-adherent dressing.

39. (canceled)

40. The device of claim 31, wherein said device further comprises one or more of a lubricant, an absorber, a sponge, a wound veil, an odor control agent, and/or a cover.

41. The device of claim 31, wherein said pyrvinium, salt or analog is contained in a liquid, salve, ointment, gel or powder disposed in or on said sterile dressing.

42. The device of claim 31, wherein said sterile dressing further comprises one or more of a corticosteroid, a cytotoxic drug, an antibiotic, an antimicrobial, an antifungal, an antiseptic, nicotine, an anti-platelet drug, an NSAID, colchicine, an anti-coagulant, a vasoconstricting drug or an immunosuppressive.

43. The device of claim 31, further comprising a port providing operable connection between said sterile dressing and a tube.

44. The device of claim 43, further comprising a cover providing for an airtight seal to or around a wound surface.

45. The device of claim 43, further comprising a drainage tube operably connected to said port at one end and suitable for attachment to a negative pressure device at another end.

46. The device of claim 43, wherein said sterile dressing is gauze or a foam.

47. (canceled)

48. A method of promoting wound repair in a subject comprising contacting said wound with a device according to claim 31.

49. The method of claim 48, wherein said method further comprises applying negative pressure to said wound.

50. The method of claim 48, wherein said method further comprises applying hyperbaric oxygen therapy to said wound, electrical stimulation, phototherapy or acoustic stimulation.

51-53. (canceled)

54. The method of claim 48, wherein said wound is a dermal wound, an epidermal wound, a burn, a laceration or abrasion, an infectious lesion, a surgical site, an ulcer, a puncture, a chronic wound, a scar, a keloid or a blister.

55-65. (canceled)

66. The method of claim 48, wherein said subject is a human.

67. The method of claim 48, wherein said subject is a non-human mammal.

68. A suture comprising pyrvinium or a salt or analog thereof impregnated into or disposed thereon.

69. The suture of claim 68, wherein said suture is an absorbable suture.

70. The suture of claim 68, wherein said suture is a liquid suture.
